(19) United States
(12) Patent Application Publication
(10) Pub. No.: US 2011/0223232 A1
(43) Pub. Date: Sep. 15, 2011

(54) DRUG-RELEASE COMPOSITION HAVING A THERAPEUTIC CARRIER

(76) Inventors: Olexander Hnojewyj, Emerald Hills, CA (US); Patrick Rivelli, JR., Palo Alto, CA (US); Tony B. Shaffer, Alameda, CA (US); David Cheung, San Jose, CA (US)

(21) Appl. No.: 12/446,498

(22) PCT Filed: Oct. 23, 2007
(86) PCT No.: PCT/US07/22540

§ 371 Date: Mar. 21, 2011

Related U.S. Application Data
(60) Provisional application No. 60/853,847, filed on Oct. 23, 2006.

(51) Int. Cl. A61F 2/82; A61K 31/60; A61K 31/603; A61K 31/192; A61K 31/196; A61K 31/405; A61K 31/407; A61K 31/436; A61P 29/00; A61P 9/00

(52) U.S. Cl. 424/423; 514/159; 514/570; 514/569; 514/567; 514/420; 514/413; 514/291

(57) ABSTRACT

The invention is generally directed to a drug-release composition that contains drug-linker-drug compound that is combined with another therapeutic agent that can be an antirestenotic agent. The therapeutic agent can be partially bound to the drug-linker-drug compound, miscible with the drug-linker-drug compound, combined with the drug-linker-drug compound at various ratios, and tuned to control the release of drugs to a tissue in need thereof.

### FIELD OF THE INVENTION

The invention is generally directed to a drug-release composition that contains drug-linker-drug compound that is combined with another therapeutic agent that can be an antirestenotic agent. The therapeutic agent can be partially bound to the drug-linker-drug compound, miscible with the drug-linker-drug compound, combined with the drug-linker-drug compound at various ratios, and tuned to control the release of drugs to a tissue in need thereof.

### BACKGROUND OF THE INVENTION

Biodegradable polymers are being used for many applications in medicine, including as a carrier for controlled-release drug delivery systems, and in biodegradable bone pins, screws, and scaffolds for cells in tissue engineering. A principal advantage of the materials based on biodegradable polymers over existing non-biodegradable polymers or metal-based material is that the products are removed over time by bioerosion, avoiding the need for surgical removal.

Despite the growing need in medical applications, only few synthetic biodegradable polymers are currently used routinely in humans as carriers for drug delivery. Poly(lactic acid) (PLA) is an example of a widely used biodegradable polymer and remains among the most important advances in medical biomaterials. However,

(1) PLA polymers typically take a few weeks to several months to completely degrade in the body and are no longer useful, as they have typically been depleted of drug more rapidly;

(2) PLA devices undergo bulk erosion, which can lead to a variety of undesirable outcomes, including exposure of unreleased drug to a highly acidic environment, and difficulty releasing drugs in a sustained manner; and,

(3) PLA does not provide a therapeutic effect in itself, either before or after biodegradation, and is limited to serving merely as a polymeric carrier for a therapeutic drug.

Such carrier materials are useful in the treatment of vascular disease. Vascular disease kills more than 16 million people worldwide each year, accounting for 30 percent of total deaths. Additional millions are disabled, frequently in their prime years. Balloon angioplasty or stenting is often used as a treatment and causes injury to the artery wall. The body responds to this injury through a natural wound healing response. There are four phases of the wound healing response: thrombosis, inflammation, proliferation, and matrix formation. In thrombosis and inflammation, there is increased production of cells and other compounds by the injured tissue, as well as the migration of inflammatory cells (such as neutrophils and macrophages) to the injured site. In the proliferation and matrix phases, smooth muscle cells found in the artery proliferate and cause the production of an extracellular matrix. Restenosis occurs when this natural healing process goes astray. Excessive levels of inflammation early on will provoke the growth of too much neointima, leading to restenosis. Excessive proliferation of smooth muscle cells will also lead to restenosis. Restenosis requires re-treatment of the patient to re-open the artery, through repeat stenting, bypass surgery, or other procedure. Late stent thrombosis occurs when the healing process is delayed or incomplete. The “open wound” can cause thrombus to collect and block the artery, leading to acute myocardial infarction with a high fatality rate.

Recent studies have suggested that patients receiving drug eluting stent (DES) treatments have a higher risk of late stent thrombosis (LST) than patients receiving BMS. Potential causes for LST are delayed and incomplete arterial healing with DES treatments, local arterial hypersensitivity reactions, and use of longer stents that create abnormal sheer stress. As permanent implants, there is also a risk that the implants may fracture and cause complications. Current stents are coated with a mixture of drug and a permanent synthetic polymer. Some researchers conclude that LST is due to hypersensitivity (severe allergy) to the synthetic polymer.

A number of biodegradable polymers are being developed, with the aim of eliminating the long-term exposure of materials within the body. However, these designs use polymers that provoke inflammation as they degrade. As a result, most biodegradable materials may cause more inflammation than a bare metal stent or a polymer coated drug-eluting stent. This inflammation leads to an increase in smooth muscle cell proliferation later in the healing process, and to excessive matrix production. The end result may be higher rates of restenosis. A common degradable polymer is poly-lactic acid (PLA), and it has been found that PLA increases inflammation in the artery after implantation and produces high rates of restenosis.

Furthermore, most biodegradable polymers degrade by “bulk erosion,” degrading both inside and outside their structure, like a sugar cube. Anti-proliferative drugs can be admixed into these polymers in an attempt to minimize restenosis. However, “bulk erosion” makes it difficult to control the release of these drugs. The polymer becomes like “swiss cheese,” and the timing of drug elution becomes uncontrollable.

The art is in need of therapeutic carriers, reducing inflammation and helping minimize restenosis. It would thus be desirable to provide a drug-release composition comprising a biocompatible material having improved biodegradation and drug-release properties, particularly if that material provides a therapeutic effect itself upon degradation. Such a material would find use in a variety of medical applications, including drug-eluting stents.

### SUMMARY OF THE INVENTION

The invention is generally directed to a drug-release composition that contains drug-linker-drug compound that is combined with another therapeutic agent that can be an antirestenotic agent. The therapeutic agent can be partially bound to the drug-linker-drug compound, miscible with the drug-linker-drug compound, combined with the drug-linker-drug compound at various ratios, and tuned to control the release of drugs to a tissue in need thereof.

In some embodiments, the invention includes a drug-release composition comprising between 50-70 percent by weight of a linked-drug compound composed of 1-10 drug-linker-drug units. The drug can be a non-steroidal anti-inflammatory drug (NSAID) having a free acid group and a second chemical group by which the drug can be coupled to the linker. The linker, in free form, should be a biocompatible molecule. In these embodiments, the compound can have anhydride linkages between drug moieties in the drug-linker-drug subunits, and between 30-50 percent by weight of an agent known to have anti-restenosis, anti-inflammatory, or anti-mitotic activity.

In some embodiments, the linker can contain from 1 to 12 carbon atoms and can be selected from a group consisting of diacids, diols, diamines, disulfides, amino acids, hydroxyalkanoates, azo compounds, diacyl dihalides, and alkyl dihalides. In these embodiments, the linker can be selected from a group consisting of oxalic acid, malonic acid, succinic acid, glutaric acid, adipic acid, pimelic acid, suberic acid, sebacic acid, azelaic acid, and their dihalide derivatives.

In many embodiments, the NSAID drug can be selected from a group consisting of salicylic acid, derivatives of salicylic acid, salsalate, diflunisal, ibuprofen, derivatives of ibuprofen, naproxen, ketoprofen, diclofenac, indomethacin, mefenamic acid, ketorolac, and iodinated salicylates. In some embodiments, the drug-linker-drug units can contain two different NSAID drugs.

In some embodiments, the compositions of the present invention can be applied to the surface of a vascular stent for release of an anti-restenosis agent and the compound drug from the stent when placed at an intravascular site. In these embodiments, the agent can be rapamycin or an analog thereof. In some embodiments, at least a portion of the rapamycin or rapamycin analog can be chemically bound to the linked-drug compound, and the rate of release of the rapamycin or rapamycin analog can be characterized by at least 60% release within 48 hours of placement of the composition in a physiological medium.

The present invention includes a method of delivering two or more drugs to a physiological release site within a patient, comprising placing at the site, a drug-release composition comprising between 50-70 percent by weight of a linked-drug compound composed of 1-10 drug-linker-drug units. In some embodiments, the drug can be a non-steroidal anti-inflammatory drug (NSAID) having a free acid group and a second chemical group by which the drug can be coupled to the linker. The linker in free form should be a biocompatible molecule. The compound can have anhydride linkages between drug moieties in the drug-linker-drug subunits, and between 30-50 percent by weight of an agent known to have anti-restenosis, anti-inflammatory, or anti-mitotic activity. In some embodiments, the release site is a site of intravascular injury, and the composition is contained on a stent deployed at the site.

These and other objects and features of the present invention will become more fully apparent when the following detailed description is read in conjunction with the accompanying figures.

### BRIEF DESCRIPTION OF THE DRAWINGS

FIG. 1 shows a stent according to some embodiments of the present invention.

FIG. 2 shows a proposed mechanism for the synthesis of a 1,3-bis(carboxyphenoyxy)propane drug-link-drug compound according to some embodiments of the present invention.

FIG. 3 illustrates the molecular weight distribution of the drug-linker-drug compound of Example 3 after preparation of the compound and after one month of aging of the compound at 37 degrees Celsius.

FIG. 4 illustrates the elution of rapamycin from a drug-release composition according to some embodiments of the present invention.

FIG. 5 shows the results of a flow cytometry study that describes the antirestenotic and anti-inflammatory properties of the drug-linker-drug compound.

FIGS. 6A and 6B compare the mean percent stenosis and intima thickness, respectively, in pig coronary arteries one month after the implant of a bare metal stent, a PLA coated stent, and a stent coated with the salicylic acid-based PAE polymer.

FIG. 7 compares cross-sectional micrographs of the mean percent stenosis and intima thickness results described in FIGS. 6A and 6B.

### DETAILED DESCRIPTION OF THE INVENTION

The compositions of the present invention include any combination of drug-linker-drug compounds and therapeutic agents; each of which can be biodegradable due to the labile nature of chemical moieties that are present. Accordingly, these compositions can be designed such that they can be broken down, absorbed, resorbed and eliminated by a mammal including, but not limited to, a human. The compositions of the present invention can be used, for example, to form pharmaceutical dosage forms, medical devices, or coatings. The terms “drug-linker-drug”, “linked-drug”, and the like, can be used interchangeably in the present application.

For the purposes of the present invention, a material is “biodegradable” when it is capable of being completely or substantially degraded or eroded when exposed to either an in vivo environment or an in vitro environment having physical, chemical, or biological characteristics substantially similar to those of the in vivo environment within a mammal. A material is capable of being degraded or eroded when it can be gradually broken-down, resorbed, absorbed and/or eliminated by, for example, hydrolysis, enzymolysis, metabolic processes, bulk or surface erosion, and the like within a mammal. It should be appreciated that traces or residue of the material may remain on a device following biodegradation. The terms “bioabsorbable” and “biodegradable” are used interchangeably in this application. In some embodiments, a bioabsorbable material has a molecular weight of 40,000 Daltons or less, 30,000 Daltons or less, 20,000 Daltons or less, 10,000 Daltons or less, 5,000 Daltons or less, or any range therein.

The drug-release compositions of the invention may be useful in many applications, such as, for example, the delivery of biologically active compounds, preparing films, coatings, medical implants, coatings for medical implants and the like. They can be readily processed into pastes or films, coatings, and microspheres with different geometric shapes. They can also be processed into finished articles or coatings using techniques known in the art, such as, for example, solvent casting, spraying solutions or suspensions, compression molding and extrusion.

Medical implant applications include vascular grafts, stents, bone plates, sutures, implantable sensors, and other articles that can completely or partially decompose over a known time period. In addition, the drug-release compositions can be used to form coatings for such articles to provide the delivery of one or more drugs to a physiological treatment site, such as vascular tissue.

The drug-release compositions of the present invention can also be incorporated into oral formulations and into products such as skin moisturizers, cleansers, pads, plasters, lotions, creams, gels, ointments, solutions, shampoos, tanning products and lipsticks for topical application.

The Drug-Release Compositions

The drug-release compositions can provide a therapeutic benefit upon biodegradation, as they contain drug-linker-drug subunit that can be biodegradable and designed to degrade at a desired rate within or between the subunits. An unprotected salicylate, for example, can be directly coupled to a diacyl dihalide, which acts as a linker to join salicylate moieties and form a drug-linker-drug subunit. The coupling of the salicylate monomers can occur in the presence of at least about 2 equivalents to about 50 equivalents of an organic base such as, for example, pyridine and the like in a suitable solvent, such as, for example, tetrahydrofuran (THF), dimethyl formamide (DMF) or mixtures thereof, to prepare the salicylate drug-linker-drug subunit for reaction with itself to form a drug-release compound containing 3-10 drug-linker-drug subunits. In some embodiments, the compound can be comprised of an average of less than 10, 9, 8, 7, 6, 5, 4, 3, or 2 drug-linker-drug subunits, and any combination thereof.

The therapeutic effect can be modulated by controlling the rate of drug release, which can be controlled by the design of the drug carrier, such that the degradation, or lack thereof, and the resulting morphology of the carrier matrix can dictate diffusion of the drug from the carrier matrix into a tissue. The character of the bonds between the drug and the carrier matrix can also affect the release rate of the drug. For example, an anhydride bond is more labile than an ester bond, an ester bond is more labile than an amide bond, and an amide bond is more labile than an ether bond, for example. Electron donating and withdrawing groups can also be implemented in the carrier for additional control.

Chemical additive groups that affect the hydrophilicity of the carrier matrix can also be added to control diffusion by assisting in the introduction of water as a diffusion medium throughout the carrier matrix. An example of such a group is poly(ethylene glycol) (PEG), which can be added as part of the polymer as a pendant group or in-chain group. Poly(ethylene glycol) can sometimes be effective when blended with a carrier matrix.

Drugs that can be used in some embodiments of the invention include, but are not limited to, antiproliferatives, antineoplastics, antimitotics, anti-inflammatories, antiplatelets, anticoagulants, antifibrins, antithrombins, antibiotics, antiallergics, antioxidants, analgesics, anesthetics, antipyretics, antiseptics, and antimicrobials. It is to be appreciated that one skilled in the art should recognize that some of the groups, subgroups, and individual bioactive agents may not be used in some embodiments of the present invention.

Antiproliferatives include, for example, actinomycin D, actinomycin IV, actinomycin I₁, actinomycin X₁, actinomycin C₁, and dactinomycin (COSMEGEN®, Merck & Co., Inc.). Antineoplastics or antimitotics include, for example, paclitaxel (TAXOL®, Bristol-Myers Squibb Co.), docetaxel (TAXOTERE®, Aventis S.A.), methotrexate, azathioprine, vincristine, vinblastine, fluorouracil, doxorubicin hydrochloride (ADRIAMYCIN®, Pfizer, Inc.) and mitomycin (MUTAMYCIN®, Bristol-Myers Squibb Co.), and any prodrugs, metabolites, analogs, homologues, congeners, derivatives, salts and combinations thereof. Antiplatelets, anticoagulants, antifibrin, and antithrombins include, for example, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors (ANGIOMAX®, Biogen, Inc.), and any prodrugs, metabolites, analogs, homologues, congeners, derivatives, salts and combinations thereof.

Cytostatic or antiproliferative agents include, for example, angiopeptin, angiotensin converting enzyme inhibitors such as captopril (CAPOTEN® and CAPOZIDE®, Bristol-Myers Squibb Co.), cilazapril or lisinopril (PRINIVIL® and PRINZIDE®, Merck & Co., Inc.); calcium channel blockers such as nifedipine; colchicines; fibroblast growth factor (FGF) antagonists, fish oil (omega 3-fatty acid); histamine antagonists; lovastatin (MEVACOR®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide, and any prodrugs, metabolites, analogs, homologues, congeners, derivatives, salts and combinations thereof. Antiallergic agents include, but are not limited to, pemirolast potassium (ALAMAST®, Santen, Inc.), and any prodrugs, metabolites, analogs, homologues, congeners, derivatives, salts and combinations thereof.

Other bioactive agents useful in the present invention include, but are not limited to, free radical scavengers; nitric oxide donors; rapamycin; everolimus; tacrolimus; 40-O-(2-hydroxy)ethyl-rapamycin; 40-O-(3-hydroxy)propyl-rapamycin; 40-O-[2-(2-hydroxy)ethoxy]ethyl-rapamycin; tetrazole containing rapamycin analogs such as those described in U.S. Pat. No. 6,329,386; estradiol; clobetasol; idoxifen; tazarotene; alpha-interferon; host cells such as epithelial cells; genetically engineered epithelial cells; dexamethasone; and, any prodrugs, metabolites, analogs, homologues, congeners, derivatives, salts and combinations thereof.

Free radical scavengers include, but are not limited to, 2,2′,6,6′-tetramethyl-1-piperinyloxy, free radical (TEMPO); 4-amino-2,2′,6,6′-tetramethyl-1-piperinyloxy, free radical (4-amino-TEMPO); 4-hydroxy-2,2′,6,6′-tetramethyl-piperidene-1-oxy, free radical (TEMPOL), 2,2′,3,4,5,5′-hexamethyl-3-imidazolinium-1-yloxy methyl sulfate, free radical; 16-doxyl-stearic acid, free radical; superoxide dismutase mimic (SODm) and any analogs, homologues, congeners, derivatives, salts and combinations thereof. Nitric oxide donors include, but are not limited to, S-nitrosothiols, nitrites, N-oxo-N-nitrosamines, substrates of nitric oxide synthase, diazenium diolates such as spermine diazenium diolate and any analogs, homologues, congeners, derivatives, salts and combinations thereof.

The drugs which can be linked into anhydrides drug-linker-drug compounds may, in some embodiments, have a relatively low molecular weight of approximately 1,000 Daltons or less and contain a carboxylic acid group. In addition, the drug needs at least one amine, thiol, alcohol or phenol group within its structure. Examples of such low molecular weight drugs with these functional groups within their structure can be found in almost all classes of drugs including, but not limited to, analgesics, anesthetics, antiacne agents, antibiotics, synthetic antibacterial agents, anticholinergics, anticoagulants, antidyskinetics, antifibrotics, antifungal agents, antiglaucoma agents, anti-inflammatory agents, antineoplastics, antiosteoporotics, antipagetics, anti-Parkinson's agents, antipsoratics, antipyretics, antiseptics/disinfectants, antithrombotics, bone resorption inhibitors, calcium regulators, keratolytics, sclerosing agents and ultraviolet screening agents.

In some embodiments, the drugs that can be used to form a drug-containing polymer component include, but are not limited to, salicylic acid, 4-aminosalicylic acid, 5-aminosalicylic acid, 4-(acetylamino)salicylic acid, 5-(acetylamino)salicylic acid, 5-chlorosalicylic acid, salicylsalicylic acid (salsalate), 4-thiosalicylic acid, 5-thiosalicylic acid, 5-(2,4-difluorophenyl)salicylic acid (diflunisal), 4-trifluoromethylsalicylic acid, sulfasalazine, dichlofenac, penicillamine, balsalazide, olsalazine, mefenamic acid, carbidopa, levodopa, etodolac, cefaclor, and captopril.

In some embodiments, the invention includes a drug-release composition comprising between 50-70 percent by weight of a linked-drug compound composed of 1-10 drug-linker-drug units. The drug can be a non-steroidal anti-inflammatory drug (NSAID) having a free acid group and a second chemical group by which the drug can be coupled to the linker. The linker, in free form, should be a biocompatible molecule. In these embodiments, the compound can have anhydride linkages between drug moieties in the drug-linker-drug subunits, and between 30-50 percent by weight of an agent known to have anti-restenosis, anti-inflammatory, or anti-mitotic activity.

In many embodiments, the NSAID drug can be selected from a group consisting of salicylic acid, derivatives of salicylic acid, salsalate, diflunisal, ibuprofen, derivatives of ibuprofen, naproxen, ketoprofen, diclofenac, indomethacin, mefenamic acid, ketorolac, and iodinated salicylates. In some embodiments, the drug-linker-drug units can contain two different NSAID drugs.

In some embodiments, the linker can contain from 1 to 12 carbon atoms and can be selected from a group consisting of diacids, diols, diamines, disulfides, amino acids, hydroxyalkanoates, azo compounds, diacyl dihalides, and alkyl dihalides. In these embodiments, the linker can be selected from a group consisting of oxalic acid, malonic acid, succinic acid, glutaric acid, adipic acid, pimelic acid, suberic acid, sebacic acid, azelaic acid, and their dihalide derivatives.

The linker unit can be, for example, an oligomer of an ether, an amide, an ester, an anhydride, a urethane, a carbamate, a carbonate, a hydroxyalkanoate, or an azo compound. And, the linker unit can also contain an additional therapeutic drug.

The linker can be chosen to biodegrade at a desired rate or remain stable in the body, as long as it is of a size that can be safely excreted. In some embodiments, a bioabsorbable material has a molecular weight of 40,000 Daltons or less, 30,000 Daltons or less, 20,000 Daltons or less, 10,000 Daltons or less, 5,000 Daltons or less, or any range therein.

A biodegradable linker can have any interunit linkage such as, for example, an ester, an anhydride, an acetal, an amide, a urethane, a urea, a glycoside, a disulfide, and a siloxane linkage. It is to be appreciated that one skilled in the art should recognize that some of these linkages may not be used in some embodiments of the present invention. The selection of the linker functionality allows for control of the relative strength or stability of the bonds provided by the linker. Control over this relative strength or stability can, for example, allow for some control over the release of drugs from the carrier matrix.

The linker can be a substituted, unsubstituted, hetero-, straight-chained, branched, cyclic, saturated or unsaturated aliphatic radical; and a substituted or unsubstituted aromatic radical. In some embodiments, the linker can comprise from about 0 to about 50 carbon atoms, from about 2 to about 40 carbon atoms, from about 3 to about 30 carbon atoms, from about 4 to about 20 carbon atoms, from about 5 to about 10 carbon atoms, and any range therein. In other embodiments, the linker can alternately comprise a non-carbon species such as, for example, a disulfide.

In some embodiments, the linker can have a molecular weight ranging from about 25 Daltons to about 1000 Daltons, from about 50 Daltons to about 750 Daltons, from about 75 Daltons to about 500 Daltons, or any range therein. In some embodiments, the linker can have a length ranging from about 5 angstroms to about 500 angstroms, from about 15 angstroms to about 400 angstroms, from about 10 angstroms to about 250 angstroms, from about 15 angstroms to about 100 angstroms, from about 25 angstroms to about 50 angstroms, or any range therein.

In some embodiments, the linker can include amino acids. In these embodiments, the amino acids can be therapeutic peptides. The therapeutic peptides can be oligopeptides, polypeptides, or proteins. Examples of amino acid sequences that can be useful in the present invention are chemokines and chemokine analogs, such as interleukins and interferons.

Applications

The benefits of the drug-release compositions of the present invention can be exploited in several medical applications that will be readily apparent to one skilled in the art. For example, in some embodiments, the drug-release composition can be used in the formation of injectable particles having sizes in the 0.5 to 50 micron size range.

Dosages of the drugs can be determined using techniques well-known to one of skill. For example, one of skill can use in vitro or in vivo activity of a drug in animal models. The extrapolation of effective doses in mice to humans, for example, is a technique that is well-known in the art. See, for example, U.S. Pat. No. 4,938,949, which is incorporated herein by reference in its entirety. Moreover, the rates of release due to hydrolysis of a drug from the polymer should also be considered, which can vary by selection of polymer, method of administration, and route of administration, as well as by the age and condition of the patient.

In some embodiments, the drug-release compositions can be used in or on a biocompatible, biodegradable intravascular stent. Currently stents for use at intravascular sites of injury are deployed by radial expansion over a balloon catheter. The present invention provides a stent that will release a combination of drugs in a controlled manner without a residual polymer material that has no therapeutic effect. In some embodiments, the compositions of the present invention can be applied to the surface of a vascular stent for release of an anti-restenosis agent and the compound drug from the stent when placed at an intravascular site. In these embodiments, the agent can be rapamycin or an analog thereof. In some embodiments, at least a portion of the rapamycin or rapamycin analog can be chemically bound to the linked-drug compound, and the rate of release of the rapamycin or rapamycin analog can be characterized by at least 60% release within 48 hours of placement of the composition in a physiological medium.

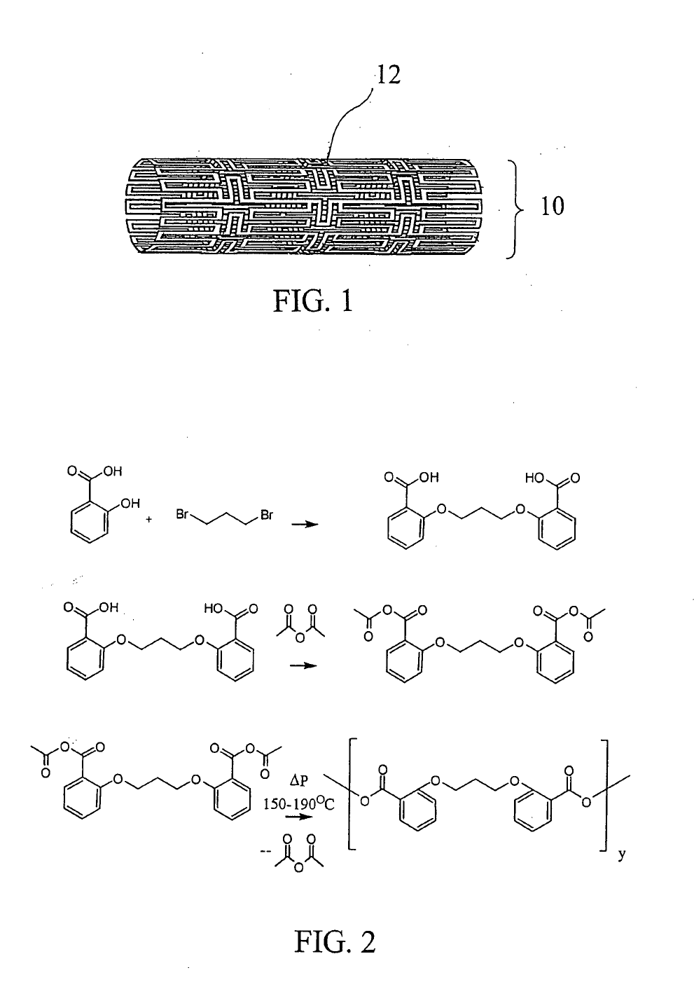

FIG. 1 shows a stent 10 according to some embodiments of the present invention. The drug-release composition can serve as a component in the stent structure or, it can be applied, for example, to all surfaces of the stent 10, or more particularly to specific surface, such as the abluminal surface 12 of the stent 10.

Typically, drug-elution is designed to occur over a relatively short period, e.g., 3 days to two weeks post implantation, and therefore the coating can be formed advantageously from a drug-release composition having rapid surface erosion characteristics. The drug-release compositions can be applied using conventional means, such as by dipping or spraying. In some embodiments, the coating has a typical thickness between 3-50 microns, 4-40 microns, 5-30 microns, 6-25 microns, 7-20 microns, 8-15 microns, or any range therein.

The drug-release compound can thus be useful in coated stent applications. In some embodiments, the invention includes a drug-release composition comprising an antirestenosis drug; and a drug-linker-drug compound having an average of no more than 10 repeating units, each repeating unit comprising two drug moieties and a linker. In these embodiments, the number of repeating units can range from about 1-10, 2-8, 2-6, 2-5, or any range therein. Each of the two drug moieties can be independently selected and comprise a component independently selected from a group consisting of immunosuppressives, anti-inflammatories, antiproliferatives, antineoplastics, antimitotics, anti-inflammatories, antiplatelets, anticoagulants, antifibrins, antithrombins, antibiotics, antiallergenics, antioxidants, and any combination thereof. The linker in the drug-linker-drug compound can have (a) a molecular weight ranging from about 25 Daltons to about 1000 Daltons; and (b) two functional groups, each of which is independently selected from a group consisting of hydroxyl, carboxyl, amino, amido, imino, diimide, and thiol groups, wherein each of the two functional groups bonds with a drug moiety in the drug-linker-drug compound.

In many embodiments, the antirestenosis drug can include a component selected from a group consisting of rapamycin, methyl rapamycin, 42-Epi-(tetrazoylyl)rapamycin (ABT-578), everolimus, tacrolimus, pimecrolimus, paclitaxel, docetaxel, estradiol, clobetasol, idoxifen, tazarotene, and any combination thereof.

In some embodiments, the antirestenosis drug can include a component selected from a group consisting of polysaccharides, peptides, non-thrombotics, antimicrobials, nitric oxide donors, free radical scavengers, and any combination thereof. In these embodiments, the polysaccharide can include a component selected from a group consisting of carboxymethylcellulose, sulfonated dextran, sulfated dextran, dermatan sulfate, chondroitin sulfate, hyaluronic acid, heparin, hirudin, and any combination thereof. The peptide can include a component selected from a group consisting of elastin, silk-elastin, collagen, atrial natriuretic peptide (ANP), Arg-Gly-Asp (RGD), and any combinations thereof. The free radical scavenger can include a component selected from a group consisting of 2,2′,6,6′-tetramethyl-1-piperinyloxy, free radical; 4-amino-2,2′,6,6′-tetramethyl-1-piperinyloxy, free radical; 4-hydroxy-2,2′,6,6′-tetramethyl-piperidene-1-oxy, free radical; 2,2′,3,4,5,5′-hexamethyl-3-imidazolinium-1-yloxy methyl sulfate, free radical; 16-doxyl-stearic acid, free radical; superoxide dismutase mimic; and any combination thereof. The nitric oxide donor can include a component selected from the group consisting of S-nitrosothiols, nitrites, N-oxo-N-nitrosamines, substrates of nitric oxide synthase, diazenium diolates, and any combination thereof.

In many embodiments, the antirestenosis drug can include a component selected from a group consisting of imatinib mesylate, paclitaxel, docetaxel, midostaurin, and any combination thereof. In some embodiments, the antirestenosis drug can include a component selected from a group consisting of estradiol, clobetasol, idoxifen, tazarotene, and any combination thereof.

A combination of drugs can be administered using the methods of the present invention. A first drug, for example, can be (i) mixed with the drug-linker-drug compound, using the drug-linker-drug compound as a carrier matrix and released upon diffusion; and/or (ii) releasably bound to the drug-linker-drug compound with or without a linker. A method of delivering two or more drugs to a physiological release site within a patient comprises placing at the site, a drug-release composition comprising between 50-70 percent by weight of a linked-drug compound composed of 1-10 drug-linker-drug units.

In some embodiments, the drug can be a non-steroidal anti-inflammatory drug (NSAID) having a free acid group and a second chemical group by which the drug can be coupled to the linker. The linker in free form should be a biocompatible molecule. The compound can have anhydride linkages between drug moieties in the drug-linker-drug subunits, and between 30-50 percent by weight of an agent known to have anti-restenosis, anti-inflammatory, or anti-mitotic activity. In some embodiments, the release site is a site of intravascular injury, and the composition is contained on a stent deployed at the site.

The invention also includes delivering a combination of drugs that are combined with the drug-linker-drug compounds of the present invention. The antirestenosis drug can include a combination of agents selected from a group consisting of everolimus and clobetasol; tacrolimus and rapamycin; tacrolimus and everolimus; rapamycin and paclitaxel; and any combination thereof.

In many drug-eluting compositions, the drug component is relatively immiscible with the carrier, which is a polymeric carrier. In the present invention, the antirestenosis drug and the drug-linker-drug compound can be miscible with each other. In fact, in some embodiments, the antirestenosis drug and the drug-linker-drug compound can form a single-phase composition when combined.

In several embodiments, a portion of the total amount of antirestenosis drug in the composition can be chemically bound to the oligodrug through a labile bond. The chemical binding can be through ionic, hydrogen, or covalent bonds.

In some embodiments, the antirestenosis drug consists of rapamycin, a rapamycin analog, or a combination thereof; and, the drug-linker-drug compound has an average of no more than 5 repeating units, wherein the two drug moieties in the repeating units are each salicylic acid, and the linker in the repeating units is adipic acid.

The present invention includes coated stent applications in which the rapamycin or rapamycin analog composes over 30% of the coating. In these embodiments, the concentration of the rapamycin or rapamycin analog can range from 30-50%, from 35-60%, from 35-70%, from 40-80%, from 35-55%, from 40-60%, from 45-65%, from 50-70%, from 55-75%, from 60-80%, and any range therein.

The present invention includes a method for controlled release of a combination of an NSAID and an antirestenosis drug to a mammalian vascular tissue. In these embodiments, the method comprises preparing a drug-release compositions described herein. The preparing includes selecting an amount of the antirestenosis drug and adding the antirestenosis drug to the composition to provide a desired initial release of the antirestenosis drug. The method further includes contacting the composition with the mammalian tissue to provide a controlled release of the antirestenosis drug and the NSAID to the mammalian tissue. In these embodiments, the mammalian tissue can be a vascular tissue.

The present invention includes method of preventing or treating a diseased tissue in a subject. In these embodiments, the method includes delivering a drug-release composition taught herein to the tissue, wherein the delivering includes coating a stent with the composition and implanting the coated stent in contact with the tissue in the subject. As above, the tissue can be a vascular tissue, and the disease can include a restenosis and vulnerable plaque, or a combination thereof.

The following examples will illustrate various methods for synthesizing and characterizing polyanhydride polymers, in accordance with the present invention, but are in no way intended to limit the scope of the invention.

### Example 1

### Synthesis of 1,3-bis(carboxyphenoyxy)propane drug-link-drug compound

### Preparation of 1,3-bis(o-carboxyphenoxy)propane Dicarboxylic Acid

FIG. 2 shows a proposed mechanism for the synthesis of a 1,3-bis(carboxyphenoyxy)propane drug-link-drug compound. 1,3-dibromopropane (14.7 mL, 0.145 mole) is added to a mixture of salicylic acid (40.0 g, 0.290 mole), distilled water (44 mL) and sodium hydroxide (23.2 g, 0.580 mole). The reaction is brought to reflux temperature before the 1,3-dibromopropane is added drop-wise. After 4 hours, additional sodium hydroxide (5.79 g, 0.145 mole) is added to the reaction mixture. Reflux is continued for another 4 hours, after which the mixture is cooled, filtered and washed with methanol.

### Preparation of poly(1,3-bis(o-carboxyphenoxy)propane)

The dicarboxylic acid was acetylated in an excess of acidic anhydride at reflux temperature. The resulting monomer was precipitated with methylene chloride into an excess of diethyl ether. The acetylated dicarboxylic acid was then allowed to react with itself in a melt condensation performed at 180 degrees Celsius for 3 hours under vacuum in a reaction vessel with a side arm. The vessel was flushed with nitrogen at frequent intervals, and the drug-linker-drug compound was isolated by precipitation into diethyl ether from methylene chloride. Reaction conditions can be chosen to obtain a desired molecular weight range.

The steps in this example can be used in the same or similar manner for drugs such as, for example, salicylates and cyclooxygenase (COX) inhibitors such as, for example, those taught in U.S. Pat. Nos. 6,468,519 and 6,685,928, each of which is hereby incorporated herein by reference in its entirety.

### Example 2

### Synthesis of a Drug-Link-Drug Compound Having a Linker that Forms Amide Bonds

One of skill will appreciate that many drugs are suitable for producing the drug-linker-drug compounds of the present invention. Other drugs that can also be protected to form amide bonds with an acyl halide linker include, but are not limited to, cephalexin, carbidopa, levodopa, and amtenac.

Amoxicillin has an amine functionality that can be used to form a drug-link-drug compound with a linker, after protecting groups have been added to the carboxyl and hydroxyl functionalities of the amoxicillin. A diacyl dihalide can be used as the linker, for example, to form amide linkages, which are less labile than ester linkages. The carboxylic acid group of the low molecular weight drug molecule is first protected, preferably via acetylation. The protected drug molecules can then be exposed to a halogenated linker, such as a brominated or chlorinated form of the linker.

The drug and linker are then exposed to heat and/or vacuum to remove the protecting groups, thereby resulting in a drug-linker-drug compound. The procedure used for amoxicillin can be used for cephalexin. The carboxylic acid of cephalexin is first protected, for example with a benzylic group. The drug is then linked to sebacyl chloride. Following this linkage, the protecting groups are removed to produce carboxylic acids which are then acetylated to produce monomer. The monomer is reacted with itself in a melt-reaction. Examples of such syntheses are known in the art and can be found, for example, in U.S. Pat. No. 6,486,214, which is incorporated herein by reference in its entirety.

### Example 3

### Synthesis of 1,6-bis(carboxyphenoyxy)hexane drug-release compound

### Preparation of 1,6-Bis(o-Carboxyphenoxy)Hexane Dicarboxylic Acid

To a mixture of salicylic acid (77.12 g, 0.5580 mole) and distilled water (84 mL) sodium hydroxide (44.71 g, 1.120 mole) is added. The reaction is brought to reflux temperature before 1,6-dibromohexane (45.21 g, 0.2790 mole) is added drop-wise. Reflux is continued for 23 hours after which additional sodium hydroxide (11.17 g, 0.2790 mole) is added. The mixture is refluxed for 16 more hours, cooled, filtered, and washed with methanol.

### Preparation of 1,6-Bis(o-Carboxyphenoxy)Hexane Monomer

The dicarboxylic acid is acetylated in an excess of acidic anhydride at reflux temperature. The resulting monomer is precipitated with methylene chloride into an excess of diethyl ether.

### Preparation of Poly(1,6-Bis(o-Carboxyphenoxy)Hexane)

The monomer is reacted with itself in a melt condensation performed at 180 degree Celsius for 3 hours under vacuum in a reaction vessel with a side arm. The reaction vessel is flushed with nitrogen at frequent intervals. The polymer is isolated by precipitation into diethyl ether from methylene chloride.

The monomers can be reacted at a variety of temperatures, for example, temperatures ranging from 100 to 300 degrees Celsius in order to vary molecular weight and polydispersity.

The hydrolysis products of these compounds are chemically similar to aspirin, which is an anti-inflammatory agent derived from salicylic acid. Therefore, the degradation products of this polymer actually aid in patient recovery.

The compounds can be characterized in a variety of ways, for example, by nuclear magnetic resonance spectroscopy, GPC, differential scanning calorimetry (DSC), thermal gravimetric analysis, contact angle measurements, UV spectroscopy, mass spectroscopy, elemental analysis and high pressure liquid chromatography (HPLC).

### Example 4

### Method of Determining the Molecular Weight of the Drug-Linker-Drug Compounds

This method is used to determine molecular weight distributions of the drug-link-drug compounds using Gel Permeation Chromatography (GPC).

### Equipment:

Waters Breeze Liquid Chromatography

Solvent: Tetrahydrofuran

Flow Rate: 0.30 ml/minute.

Columns: Waters GPC Columns

The system was calibrated using polystyrene standards.

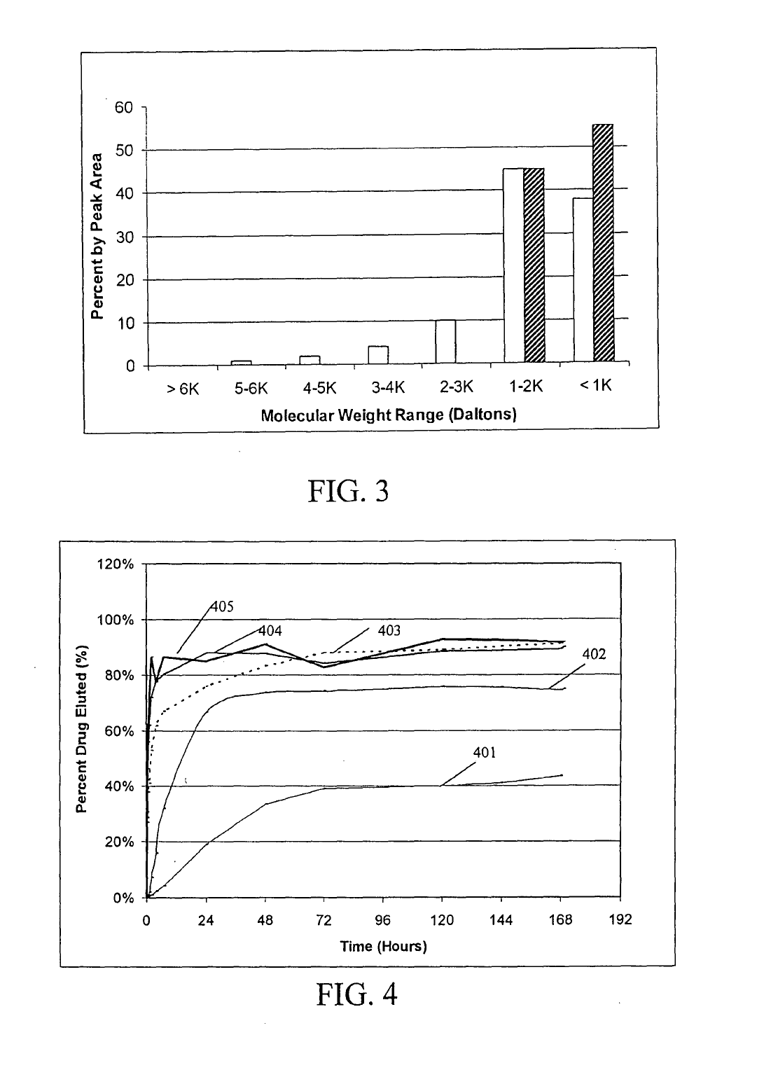

FIG. 3 illustrates the molecular weight distribution of the drug-linker-drug compound of Example 3 after preparation of the compound and after one month of aging of the compound at 37 degrees Celsius. The degradation of this compound is performed in chromatography grade water at 37° C. The samples were aged in a closed test tube. The blank bars indicate before aging, and the striped bars indicate after aging. Table 2 compares the data before and after the aging in terms of the percent contribution of the molecular weight of the compounds in each bin to the average molecular weight of the compound.

TABLE 2

| M.W. Bins | Before Aging | After Aging One Month |
|---|---|---|
| (KDaltons) | (% Contribution) | (% Contribution) |
| >6 | 0 | 0 |
| 5-6 | 1 | 0 |
| 4-5 | 2 | 0 |
| 3-4 | 4 | 0 |
| 2-3 | 10 | 0 |
| 1-2 | 45 | 45 |
| <1 | 38 | 55 |
| Average M.W. (Low End) | 904 | 456 |
| Average M.W. (High | 1900 | 1450 |
| End) | | |

### Example 5

### Differential Scanning Calorimetry of Drug-Release Compounds

Differential Scanning calorimetry was used to characterize the drug-release compositions of the present invention. The analyses were performed by Rose Consulting of Half Moon Bay, Calif.

Two drug-linker-drug compounds were combined with rapamycin at various concentrations. The first drug-linker-drug compound was a solution-reacted compound (DLD_{S}), and the second drug-linker-drug compound was a melt-reacted compound (DLD_{M}). The experiment was performed to determine the Tg profiles of the compositions, and the data is provided in Table 3.

TABLE 3

| | Tg for DLD_{S} | Tg for DLD_{M} |
|---|---|---|
| Sample | (° C.) | (° C.) |
| 85/15 run 1 | 39.56 | |
| 85/15 run 2 | 27.03 | 57.23 |
| 75/25 run 1 | 39.52 | |
| 75/25 run 2 | 41.05 | 51.27/50.04 |
| 50/50 run 1 | 46.84 | |
| 50/50 run 2 | 48.83 | 53.91/51.78 |
| 100/0 | 41.88 | 80.72 |

The samples were applied to wafers in solution form and allowed to dry overnight at 40 degrees Celsius. The samples were then scraped off the wafers and tested to 90 degrees Celsius, held for one minute and then control-cooled to 5 degrees Celsius and retested to 90 degrees Celsius. All testing was done in a dry nitrogent environment.

The data shown in Table 3 suggests a solid/solid interaction between the DLD_{M} compound and the rapamycin resulting in a decrease in the glass-transition temperature from 80.7 degrees Celsius for 100 percent drug-linker-drug down to a low of 50 degrees Celsius for the mixtures. This large drop in glass-transition temperature does not appear with the DLD_{S} compound when mixed with rapamycin, as there is little to no change in Tg when the rapamycin is added.

The data suggests that the rapamycin is miscible with the DLD_{M} compound and relatively immiscible with the DLD_{S} compound. Furthermore, it was noticed that there was fluorescence in the DLD_{M} compound and not the DLD_{S} compound, suggesting that the interaction is a conjugation of the rapamycin to the DLD_{M} compound. The most likely reaction in this system would be a conjugation between a hydrolyzed anhydride bond in the DLD_{M} compound and a free hydroxyl group in the rapamycin, resulting in the formation of an ester bond.

### Example 6

### Elution of Rapamycin from a Drug-Link-Drug Compound

This method is used to test and measure the elution profile of rapamycin from the drug-release compositions. The test utilizes a closed-cell recirculating system (SOTAX CE7 Smart USP IV Flow Through Dissolution System) to collect rapamycin as it elutes from the sample compositions. An aqueous-based extraction medium is pumped through the closed samples cells and back into a medium reservoir. The extraction solution is maintained at 37° C. during the test period. Aliquots are taken from the sample reservoir at given time intervals and the rapamycin concentration is measured using Reverse Phase High Pressure Liquid Chromatography (HPLC).

The elution profiles of the rapamcyin are generated by charting the % rapamycin eluted against the elution time.

FIG. 4 illustrates the elution of rapamycin from a drug-release composition according to some embodiments of the present invention. The drug release from five drug-release compositions having varying ratios of rapamycin and the drug-linker-drug compound of Example 3 were tested. Table 1 shows the composition ratios that were used to produce the elution profiles.

The compositions were applied to a 3.0 mm diameter, 316L stainless steel stent, 18 mm in length, to form a coated stent; and, the stent was crimped onto a balloon catheter with a Machine Solutions crimper, E-beam sterilized, and expanded to simulate actual physical stresses in the composition that may be incurred during production and use of a coated stent.

TABLE 1

| | Ratio |
|---|---|
| | (% Drug-Linker- |
| Elution Curve | Drug/% Rapamycin) |
| 401 | 85/15 |
| 402 | 75/25 |
| 403 | 70/30 |
| 404 | 65/35 |
| 405 | Cordis CYPHER Stent |

The CYPHER stent was used for comparison, as it is composed of three layers of polymers over a frame made of laser cut 316L stainless steel, electropolished, and coated in a primer layer of Parylene C. A mixture of polyethylene-co-vinyl acetate (PEVA) and poly n-butyl methacrylate (PBMA) is then dissolved in THF. The ratio of PBMA to PEVA is 67% PEVA, 33% PBMA. Sirolimus (rapamycin) is then dissolved in the THF/polymer mixture and the mixture is applied to the Parylene C coated stent. Another mixture of PEVA and PBMA, without sirolimus, is dissolved in THF and applied to the stent by spraying with a fine nozzle. This outer coating prevents the so-called “burst effect” which results when drug on the surface of the polymer is rapidly released following immersion in water or another solvent. A small amount of sirolimus migrates to the final layer during the formation of the outer layer because it dissolves in the THF and precipitates in the PEVA/PBMA outer layer. The small amount of sirolimus in the outer layer has a small but noticeable burst effect. The entire three layered coating is applied to both the luminal and abluminal sides of the stainless steel stent. Finally, the stent is placed on a delivery catheter, sterilized, and packaged.

The drug-release composition of the present invention has a controllable release profile, even when present as a single layer with no underlayer or outer layer. The elution profiles show that the amount of rapamycin released from the drug-release composition in the early stages of implantation increases with an increase in the concentration of rapamycin. The initial release is followed by a long term, sustained release of rapamycin as the drug-linker-drug compound degrades and releases the NSAID.

### Example 7

### Non-Inflammatory Effect of a Drug-Linker-Drug Compound Containing an NSAID

The drug-linker-drug compound containing salicylic acid has substantial antirestenotic and anti-inflammatory properties. A biodegradable polyanhydride ester polymer available from Bioabsorbable Therapeutics, Inc. of Menlo Park, Calif. The polymer can have the general form,

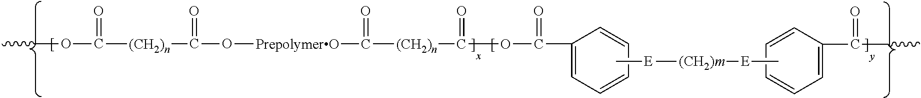

The E can be an ortho, meta, or para ester or ether linkage, and the pre-polymer can be a dihydroxy terminated polyester or polyether polymer having a molecular weight in a selected range from about 1 to about 10 Kdaltons. The x can be about 80% to about 98% of the polymer by weight, the y (drug-linker-drug compound) can be about 20% to about 2% of the polymer by weight, the n can range from about 2 to about 4, the m can range from about 2 to about 10; and the average total number of anhydride linkages can be a selected number ranging from about 5 to about 30.

In the polymer of the present example (“the salicylic acid-based PAE polymer”), the drug-linker-drug compound is salicylic acid-adipic acid-salicylic acid, and the prepolymer is PLA. Although the present invention is directed to a composition comprising the drug-linker-drug compound and not this polymer, the results obtained from this polymer-based experiment are representative of the therapeutic antirestenotic and anti-inflammatory properties that can be expected from the NSAID-containing drug-linker-drug compound.

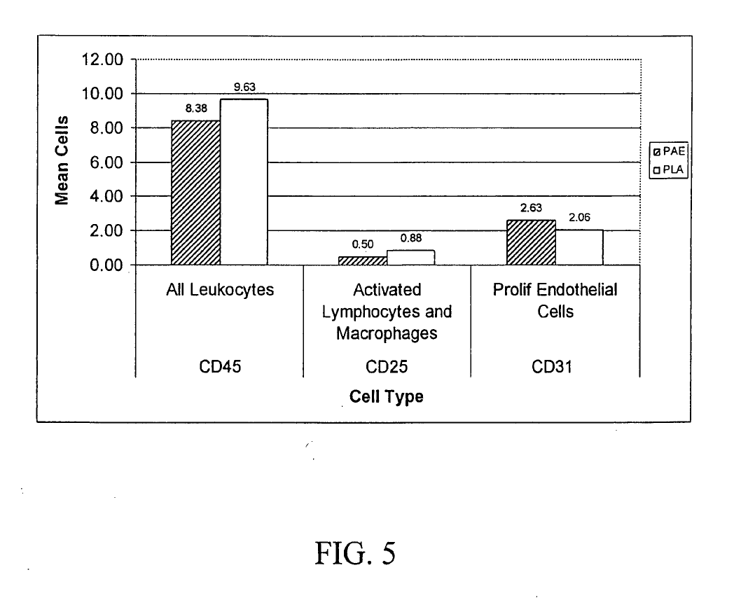

The therapeutic antirestenotic and anti-inflammatory properties of the salicylic acid-based PAE polymer was analyzed using a porcine coronary model for flow cytometry. FIG. 5 shows the results of a flow cytometry study that describes the antirestenotic and anti-inflammatory properties of the drug-linker-drug compound. The flow-cytometry study compared measurements of acute inflammation three days after implantation of a metal stent coated with PLA only and a metal stent coated with the salicylic acid-based PAE polymer in a porcine coronary artery model. The salicylic acid-based PAE polymer showed significantly less inflammation, as measured by CD45 cells (all leukocytes) and CD25 cells (activated lymphocytes and macrophages). The salicylic acid-based PAE polymer also had more CD31 cells (proliferating endothelial cells), which is an indication of a superior healing response.

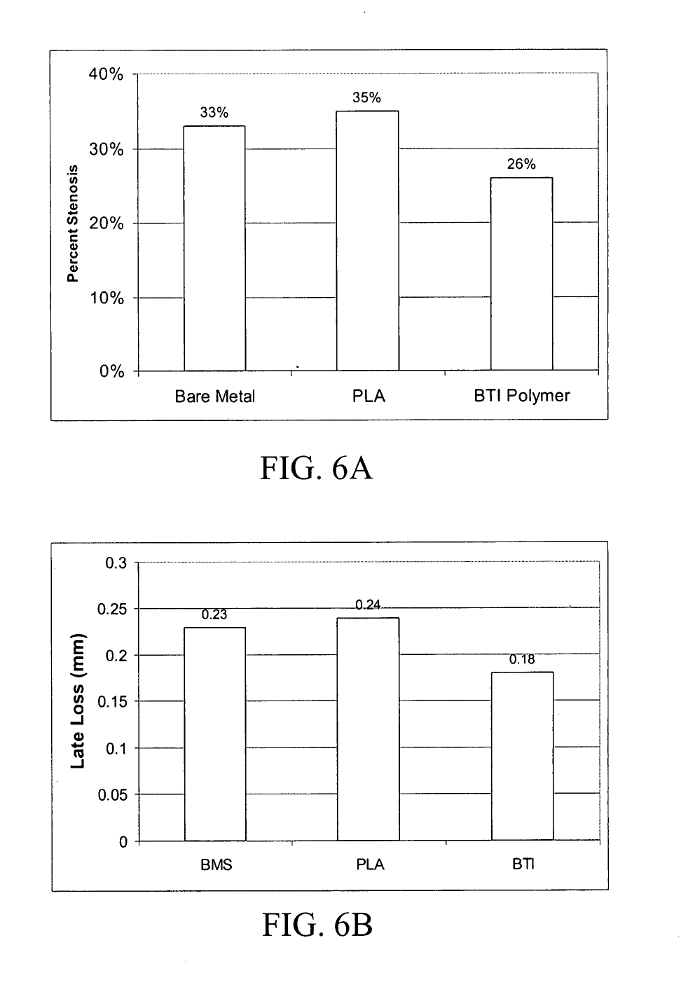

FIGS. 6A and 6B compare the mean percent stenosis and intima thickness, respectively, in pig coronary arteries one month after the implant of a bare metal stent, a PLA coated stent, and a stent coated with the salicylic acid-based PAE polymer. The PLA coated stent had a higher mean percent stenosis and a higher late loss than the bare metal stent, suggesting that the increase in acute inflammation from the PLA can lead to an increase in stenosis.

Late loss is defined as the difference between immediate post-procedure minimum lumen diameter (MLD) and the MLD 6 to 9 months after a percutaneous coronary intervention. It is an angiographic measure of the absolute amount of renarrowing and an accepted way of determining the probability of restenosis. Specifically, late loss measures the change in MLD of the treated coronary segment due to vascular contraction and neointimal hyperplasia. For coronary stents, which by design resist vascular contraction and generally achieve a uniform lumen diameter within the stented segment, late loss is an angiographic surrogate for neointimal hyperplasia, the target of drug-eluting stents.

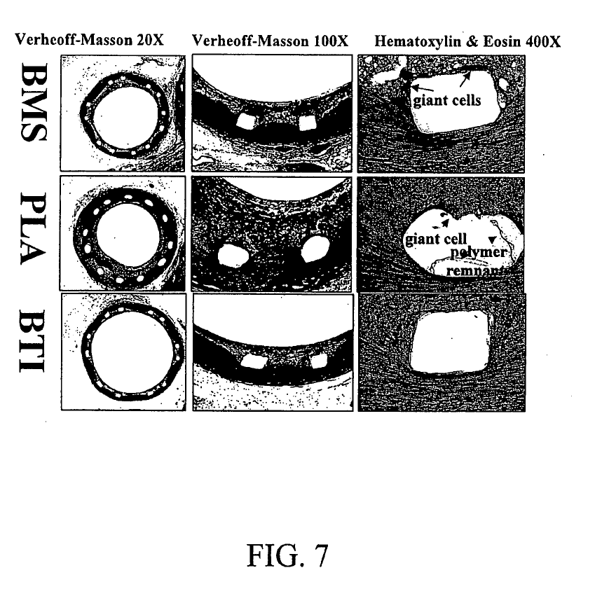

The salicylic acid-based PAE polymer had a lower mean percent stenosis and lower late loss than the bare metal stent, suggesting that the anti-inflammatory effect of the salicylic acid may reduce stenosis. FIG. 7 compares cross-sectional micrographs of the mean percent stenosis and intima thickness results described in FIGS. 6A and 6B.

Although the invention has been described with respect to certain methods and applications, it will be appreciated that a variety of changes and modification may be made without departing from the invention as claimed. The use of a chemical in a class is intended to represent the use of any chemical in that class. The listing of a Markush group can be construed, as will be apparent to one of skill, to include all members of the group or exclude any member of the group, depending on the particular embodiment.

It is claimed:

1. A drug-release composition comprising:
between 50-70 percent by weight of a linked-drug compound composed of 1-10 drug-linker-drug units, where the drug is a non-steroidal anti-inflammatory drug (NSAID) having a free acid group and a second chemical group by which the drug can be coupled to the linker, the linker in free form is a biocompatible molecule, and the compound has anhydride linkages between drug moieties in the drug-linker-drug subunits, and
between 30-50 percent by weight of an agent known to have anti-restenosis, anti-inflammatory, or anti-mitotic activity.

2. The composition of claim 1, wherein the linker contains from 1 to 12 carbon atoms and is selected from a group consisting of diacids, diols, diamines, disulfides, amino acids, hydroxyalkanoates, azo compounds, diacyl dihalides, and alkyl dihalides.

3. The composition of claim 1, wherein the linker is selected from a group consisting of oxalic acid, malonic acid, succinic acid, glutaric acid, adipic acid, pimelic acid, suberic acid, sebacic acid, azelaic acid, and their dihalide derivatives.

4. The composition of claim 1, wherein the NSAID drug is selected from a group consisting of salicylic acid, derivatives of salicylic acid, salsalate, diflunisal, ibuprofen, derivatives of ibuprofen, naproxen, ketoprofen, diclofenac, indomethacin, mefenamic acid, ketorolac, and iodinated salicylates.

5. The composition of claim 1, wherein the drug-linker-drug units contain two different NSAID drugs.

6. The composition of claim 1, which is applied to the surface of a vascular stent for release of an anti-restenosis agent and the compound drug from the stent when placed at an intravascular site.

7. The composition of claim 6, wherein said agent is rapamycin or an analog thereof.

8. The composition of claim 7, wherein at least a portion of the rapamycin or rapamycin analog is chemically bound to the linked-drug compound, and the rate of release of the rapamycin or rapamycin analog is characterized by at least 60% release within 48 hours of placement of the composition in a physiological medium.

9. A method of delivering two or more drugs to a physiological release site within a patient, comprising placing at the site, a drug-release composition comprising
between 50-70 percent by weight of a linked-drug compound composed of 1-10 drug-linker-drug units, where the drug is a non-steroidal anti-inflammatory drug (NSAID) having a free acid group and a second chemical group by which the drug can be coupled to the linker, the linker in free form is a biocompatible molecule, and the compound has anhydride linkages between drug moieties in the drug-linker-drug subunits, and
between 30-50 percent by weight of an agent known to have anti-restenosis, anti-inflammatory, or anti-mitotic activity.

10. The method of claim 9, wherein the release site is a site of intravascular injury, and the composition is contained on a stent deployed at the site.

11. The method of claim 10, wherein
the linker is selected from the group consisting of oxalic acid, malonic acid, succinic acid, glutaric acid, adipic acid, pimelic acid, suberic acid, sebacic acid, azelaic acid, and their dihalide derivatives;
the NSAID drug is selected from a group consisting of salicylic acid, derivatives of salicylic acid, salsalate, diflunisal, ibuprofen, derivatives of ibuprofen, naproxen, ketoprofen, diclofenac, indomethacin, mefenamic acid, ketorolac, and iodinated salicylates; and,
the antirestenosis agent is rapamycin or an analog thereof.

12. The method of claim 11, wherein at least a portion of the rapamycin or rapamycin analog is covalently linked to the linked-drug compound via ester linkages, and the rate of release of the rapamycin or rapamycin analog is characterized by at least 60% release within 48 hours of placement of the composition in a physiological medium.
